(19) United States
(12) Patent Application Publication
(10) Pub. No.: US 2021/0228539 A1
(43) Pub. Date: Jul. 29, 2021

(54) SOLUBLE EPOXIDE HYDROLASE AS A TARGET FOR OCULAR DISEASES

(71) Applicant: Indiana University Research and Technology Corporation, Indianapolis, IN (US)

(72) Inventors: Timothy William Corson, Fishers, IN (US); Rania Sulaiman, Indianapolis, IN (US)

(21) Appl. No.: 17/226,814

(22) Filed: Apr. 9, 2021

Related U.S. Application Data
(63) Continuation of application No. 16/538,175, filed on Aug. 12, 2019.

(51) Int. Cl. A61K 31/353; A61P 27/02; A61K 9/00; A61K 31/423; A61K 39/395; A61K 31/192; A61K 9/06

(52) U.S. Cl. CPC A61K 31/353; A61P 27/02; A61K 9/0048; A61K 9/0019; A61K 9/06; A61K 9/0053; A61K 39/3955; A61K 31/192; A61K 31/423

(57) ABSTRACT

Methods of using compounds to inhibit ocular disease are disclosed herein. Methods are disclosed for inhibiting soluble epoxide hydrolase (sEH) for the treatment of ocular diseases, and in particular, wet age-related macular degeneration (AMD).

### CROSS REFERENCE TO RELATED APPLICATIONS

This application is a continuation application of U.S. application Ser. No. 16/538,175, filed Aug. 12, 2019, which is a divisional application of U.S. application Ser. No. 15/889,464, filed Feb. 6, 2018, which claims the benefit to U.S. Provisional Patent Application No. 62/458,322, filed on Feb. 13, 2017, both of which are hereby incorporated by reference in their entireties.

### STATEMENT OF GOVERNMENT SUPPORT

This invention was made with government support under EY025641 and TR001106 awarded by the National Institutes of Health. The government has certain rights in the invention.

### BACKGROUND OF THE DISCLOSURE

The present disclosure relates generally to the use of compounds for inhibition of soluble epoxide hydrolase (sEH) for the treatment of diseases. In one embodiment, the compound for inhibition of sEH is the anti-angiogenic small molecule, SH-11037. More particularly, the present disclosure relates to the use of sEH inhibitors for the treatment of ocular diseases, and in particular, of wet age-related macular degeneration (AMD).

Ocular neovascularization is the underlying cause of blindness in diseases such as retinopathy of prematurity (ROP), proliferative diabetic retinopathy (PDR), and wet age-related macular degeneration (AMD), which affect infants, adults of working age and the elderly, respectively. Currently, the gold standard, FDA approved treatments for wet AMD focus on inhibiting the vascular endothelial growth factor (VEGF) signaling pathway using biologics such as ranibizumab and aflibercept. Despite the success of these medications, their association with ocular and systemic side effects due to inhibition of such a major angiogenic pathway, and the presence of resistant and refractory patient populations, complicate their use.

Based on the foregoing, there is a crucial need in the art for new therapeutic targets for ocular diseases. It has been found herein that alternative angiogenic targets could form the basis for new therapeutics to complement and combine with existing medications.

### BRIEF DESCRIPTION

The present disclosure is generally related to the use of compounds for the inhibition of soluble epoxide hydrolase (sEH), and to methods of inhibiting sEH for treating diseases, and particularly, for treating ocular diseases. In one aspect, the present disclosure further characterizes a previously identified antiangiogenic homoisoflavonoid derivative, SH-11037 (1; FIG. 1A), previously shown to have potent antiangiogenic activities in vivo in the laser-induced choroidal neovascularization (L-CNV) mouse model. In the present disclosure, SH-11037 (1) was found to effectively inhibit soluble epoxide hydrolase (sEH) in vitro and in vivo.

In another aspect, the present disclosure has surprisingly found that sEH levels are dramatically upregulated in ocular sections from a choroidal neovascularization (CNV) mouse model and human wet AMD eyes compared to controls. Further, known sEH inhibitors significantly suppressed CNV vascular volume in mice in a dose-dependent manner. Based on these results, the present disclosure has identified sEH as a target for inhibiting ocular diseases, and in particular, wet AMD.

Accordingly, in one aspect, the present disclosure is directed to a method of inhibiting soluble epoxide hydrolase (sEH) in a subject in need thereof, the method comprising administering to the subject an amount of SH-11037 (1)

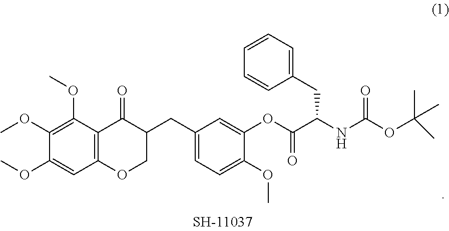 [SMILES: COc1ccc(CC2COc3cc(OC)c(OC)c(OC)c3C2=O)cc1OC(=O)[C@H](Cc1ccccc1)NC(=O)OC(C)(C)C]

In another aspect, the present disclosure is directed to a method of inhibiting ocular disease in a subject in need thereof, the method comprising administering to the subject a soluble epoxide hydrolase (sEH) inhibitor selected from the group consisting of 7-(trifluoromethyl)-N-(4-(trifluoromethyl)phenyl) benzo[d]isoxazol-3-amine (7); 12-(3-((3s,5s,7s)-adamantan-1-yl)ureido)dodecanoic acid (AUDA); sorafenib; 1-(1-acetyl-piperidin-4-yl)-3-adamantan-1-yl-urea (AR9281); (1R,3S)—N-(4-cyano-2-(trifluoromethyl)benzyl)-3-((4-methyl-6-(methylamino)-1,3,5-triazin-2-yl)amino)cyclohexane-1-carboxamide (GS K2256294); trans-4-{4-[3-(4-trifluoromethoxy-phenyl)-ureido]-cyclohexyloxy}-benzoic acid (t-TUCB or UC1728); N-[(1S,2R)-2-phenylcyclopropyl]-4-[3-(2-pyridinyl)-1,2,4-oxadiazol-5-yl]-)1-piperidinecarboxamide; antisense RNA targeting sEH (EPHX2) RNA; shRNA targeting sEH (EPHX2) RNA; siRNA targeting sEH (EPHX2) RNA; RNA silencing targeting sEH (EPHX2) RNA; RNA interference (RNAi) targeting sEH (EPHX2) RNA; CRISPR/Cas9-mediated genetic ablation of sEH (EPHX2) genomic DNA; zinc-finger nuclease-mediated genetic ablation of sEH (EPHX2) genomic DNA; and combinations thereof.

In another aspect, the present disclosure is directed to a method of treating wet age-related macular degeneration (AMD) in a subject, the method comprising administering to the subject a soluble epoxide hydrolase (sEH) inhibitor. The sEH inhibitor is one or more of 7-(trifluoromethyl)-N-(4-(trifluoromethyl)phenyl) benzo[d]isoxazol-3-amine (7); 12-(3-((3s,5s,7s)-adamantan-1-yl)ureido)dodecanoic acid (AUDA); sorafenib; 1-(1-acetyl-piperidin-4-yl)-3-adamantan-1-yl-urea (AR9281); (1R,3S)—N-(4-cyano-2-(trifluoromethyl)benzyl)-3-((4-methyl-6-(methylamino)-1,3,5-triazin-2-yl)amino)cyclohexane-1-carboxamide (GSK2256294); trans-4-{4-[3-(4-trifluoromethoxy-phenyl)-ureido]-cyclohexyloxy}-benzoic acid (t-TUCB or UC1728); N-[(1S,2R)-2-phenylcyclopropyl]-4-[3-(2-pyridinyl)-1,2,4-oxadiazol-5-yl]-)1-piperidinecarboxamide; antisense RNA targeting sEH (EPHX2) RNA; shRNA targeting sEH (EPHX2) RNA; siRNA targeting sEH (EPHX2) RNA; RNA silencing targeting sEH (EPHX2) RNA; RNA interference (RNAi) targeting sEH (EPHX2) RNA; CRISPR/Cas9-mediated genetic ablation of sEH (EPHX2) genomic DNA; zinc-finger nuclease-mediated genetic ablation of sEH (EPHX2) genomic DNA.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIGS. 1A-1C depict soluble epoxide hydrolase (sEH) as a target of antiangiogenic homoisoflavonoid SH-11037. (FIG. 1A) Structures of SH-11037 (1), ester affinity reagent (2), amide affinity reagent (3), and negative control reagent (4). BP, benzophenone moiety. (FIG. 1B) Proteins pulled down with indicated reagents were separated by SDS-PAGE and silver stained, then identified by mass spectrometry. A unique band was present in pulldown with 3 but not 4, box; asterisks represent non-specific bands. (FIG. 1C) Immunoblot of pulled down protein using antibody against sEH. Silver-stained gel and immunoblot are representatives from at least two independent experiments.

FIGS. 2A-2D depict SH-11037 is an sEH inhibitor. (FIG. 2A) SH-11037 (1), but not its inactive analog SH-11098 (6) significantly suppressed sEH enzymatic activity in vitro, IC₅₀=0.15 μM (SH-11098 IC₅₀>10 μM). The specific sEH inhibitors t-AUCB (5) and compound 7 were used as positive controls, IC₅₀=9.5 nM for each. Mean±SEM from triplicate wells shown. (FIG. 2B) Michaelis-Menten kinetic response plot for sEH-mediated hydrolysis of fluorogenic substrate, PHOME, for varying SH-11037 concentrations. Mean±SEM from triplicate wells shown. (FIG. 2C) Lineweaver-Burk plot of these data suggested mixed-type inhibition. (FIG. 2D) Dixon plot further supports mixed-type inhibition.

FIGS. 2E-2G depict kinetic analysis for sEH inhibition by compound 7. (FIG. 2E) Michaelis-Menten kinetic response plot for hydrolysis of fluorescent substrate, PHOME. (FIG. 2F) Lineweaver-Burk plot indicates mixed-type inhibition. (FIG. 2G) Kᵢ=19.6±5.4 nM is illustrated on Dixon plot. Mean±SEM, n=3. Representative results from at least two independent experiments.

FIGS. 2H & 2I are secondary plots for enzyme kinetic analyses. The apparent K_{M}/Vₘₐₓ data fit the expected profiles for mixed-type inhibition by SH-11037 and 7. (FIG. 2H) K_{Mapp}/Vₘₐₓₐₚₚ and 1/Vₘₐₓₐₚₚ vs. [SH-11037]. (FIG. 2I) K_{Mapp}/Vₘₐₓₐₚₚ and 1/Vₘₐₓₐₚₚ vs. [7]. Representative results from at least two independent experiments.

FIG. 2J depicts the lipid profile of retina/choroid for DHA-related metabolites from L-CNV (3 days post-laser-treatment) or control mice treated with vehicle, 10 μM t-AUCB, or 10 μM SH-11037. EDP, epoxydocosapentaenoic acids; DHDP, dihydroxydocosapentaenoic acids.

FIG. 2K shows the ratio of 19,20 EDP/DHDP between different treatment conditions and vehicle only (no laser) control mice indicates increased sEH levels and activity 3 days following laser induction compared to no laser control, **P<0.01, and a significant sEH inhibition by SH-11037, *P<0.05 vs vehicle. One-way ANOVA, Dunnett's post hoc tests. Mean±SEM, n=5 mice/treatment. Activity assay and kinetic analyses are representatives from at least two independent experiments.

FIGS. 3A-3E show that sEH is upregulated in the eyes of mice and humans undergoing neovascularization. (FIG. 3A) Representative images of retinal sections from laser-induced choroidal neovascularization (L-CNV) and control eyes stained with DAPI (blue), agglutinin for vasculature (green), and sEH (magenta), showing upregulation of sEH in the outer retina in L-CNV sections 3 days post-laser treatment. (FIG. 3B) Immunoblot of sEH protein levels in mouse retina and choroid sections of laser treated mouse eyes compared to untreated controls; β-actin is a loading control. Pooled eyes from two independently treated animals per condition. (FIG. 3C) Representative images of retinal sections from L-CNV (3 days post laser-treatment) and control eyes stained with DAPI (blue), sEH (magenta), and rod marker rhodopsin (green), showing co-localization of upregulated sEH with rod photoreceptors. (FIG. 3D) sEH activity is up-regulated in L-CNV eye tissue (*P<0.05) and normalized by 20 μM SH-11037 or 7 treatment (***P<0.001), as indicated in a trans-stilbene oxide enzymatic activity assay performed 3 days post-laser-treatment. Mean±SEM, ANOVA with Tukey's post hoc tests. Pooled data from three experiments, n=2-3 animals per condition per experiment. (FIG. 3E) Representative images of central retinal sections from eyes of human wet AMD patients (78 years old) and age-matched controls (68 years old). sEH is magenta, vasculature (FITC-agglutinin) is green, and nuclei (DAPI) are blue. In wet AMD, sEH is increased in the inner retina and aberrantly expressed in some photoreceptors (arrowheads). Scale bars=50 μm. IgG is a negative control with preimmune primary antibodies. GCL=ganglion cell layer; INL=inner nuclear layer; ONL=outer nuclear layer; IS/OS, photoreceptor inner segments/outer segments; RPE, retinal pigment epithelium.

FIG. 4 is a further example of differential sEH staining in the central retina of human wet AMD eyes (68 years old) versus age-matched controls (78 years old). sEH is magenta, vasculature (FITC-agglutinin) is green, and nuclei (DAPI) are blue. In wet AMD, sEH is increased in the inner retina, and aberrantly expressed in some photoreceptors (arrowheads). GCL, ganglion cell layer; INL, inner nuclear layer; ONL, outer nuclear layer; IS/OS, photoreceptor inner segments/outer segments; RPE, retinal pigment epithelium. Scale bars=50 μm.

FIG. 5 shows further evidence of sEH upregulation within L-CNV lesions 3 days post laser-treatment, and co-staining with rod (rhodopsin) but not cone (arrestin) markers. sEH is magenta, cell type markers are green, and nuclei (DAPI) are blue. GCL, ganglion cell layer; INL, inner nuclear layer; ONL, outer nuclear layer; IS/OS, photoreceptor inner segments/outer segments; RPE, retinal pigment epithelium. Scale bars=50 μm.

FIGS. 6A & 6B show that sEH does not colocalize with markers of (FIG. 6A) horizontal cells (calbindin) or (FIG. 6B) retinal ganglion cells (Brn3a) in untreated C57BL/6 adult mouse eyes or in L-CNV eyes 3 days post laser-treatment. sEH is magenta, cell type markers are green, and nuclei (DAPI) are blue. GCL, ganglion cell layer; INL, inner nuclear layer; ONL, outer nuclear layer; IS/OS, photoreceptor inner segments/outer segments; RPE, retinal pigment epithelium. Scale bars=50 μm.

FIGS. 7A & 7B show that sEH does not colocalize with markers of (FIG. 7A) cone photoreceptors (cone arrestin) or (FIG. 7B) Müller glia (vimentin) in untreated C57BL/6 adult mouse eyes or in L-CNV eyes, 3 days post laser-treatment. Inset shows magnification of area marked by dotted lines. sEH is magenta, cell type markers are green, and nuclei (DAPI) are blue. GCL, ganglion cell layer; INL, inner nuclear layer; ONL, outer nuclear layer; IS/OS, photo receptor inner segments/outer segments; RPE, retinal pigment epithelium. Scale bars=50 μm.

FIGS. 8A-8F show that local application of sEH inhibitors dose-dependently suppresses neovascularization. (FIG. 8A) Representative images from confocal microscopy of agglutinin stained CNV lesions 14 days post-laser-treatment, scale bar=50 μm. (FIGS. 8B & 8C) Dose-dependent inhibition of the volume of CNV lesions by (FIG. 8B) t-AUCB (5) and (FIG. 8C) sEH inhibitor 7 compared to vehicle control. Mean±SEM, n=6-15 animals/treatment (one eye per animal). * P<0.05, **P<0.01, ***P<0.001 compared to vehicle, one-way ANOVA, Dunnett's post hoc tests. (FIGS. 8D & 8E) Inhibition of mouse choroidal sprouting ex vivo by (FIG. 8D) t-AUCB (5) and (FIG. 8E) sEH inhibitor 7 compared to vehicle control. Mean±SEM, n=4 eyes/treatment, representative data from at least two independent experiments. Axes for measurement of sprouting distance shown in yellow. Scale bars=1 mm. *P<0.05, **P<0.01 compared to vehicle, one-way ANOVA, Dunnett's post hoc tests. (FIG. 8F) Summary of SH-11037's mechanism. By inhibiting sEH, SH-11037 decreases the formation of 19,20-DHDP (dihydroxydocosapentaenoic acid), and increases levels of docosahexaenoic acid (DHA)-derived 19,20-EDP (epoxydocosapentaenoic acid), with antiangiogenic effects.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Soluble epoxide hydrolase (sEH, encoded by EPHX2) is a 62 kDa bifunctional enzyme that has N-terminal lipid phosphatase (EC 3.1.3.76) and C-terminal epoxide hydrolase (EC 3.3.2.10) activities. While the physiological role of the lipid phosphatase activity of sEH is not fully understood, its epoxide hydrolase activity has been extensively studied due to its role in the metabolism of arachidonic acids' epoxide intermediates, epoxyeicosatrienoic acids (EETs). While EETs have been shown to have proangiogenic effects resulting in accelerating tumor growth, they play a role in hypertension, pain and inflammation. Additionally, sEH is also involved in the metabolism of the epoxides of ω-3 fatty acids, docosahexaenoic acid (DHA) and eicosapentaenoic acid (EPA).

In one embodiment, the present disclosure has identified an inhibitor of sEH. Particularly, the antiangiogenic homoisoflavonoid derivative, SH-11037 (1), has been found to inhibit sEH in vitro and in vivo.

The method for synthesizing (2S)-2-methoxy-5-((5,6,7-trimethoxy-4-oxochroman-3-yl)methyl)phenyl 2-((tert-butoxycarbonyl)amino)-3-phenylpropanoate (1, SH-11037), is disclosed in PCT Publication No. WO2014/182695, which is hereby incorporated by reference to the extent it is consistent herewith.

In another aspect of the present disclosure, it has been found that inhibitors of soluble epoxide hydrolase (sEH), including SH-11037 (1), can be used for treatments of various diseases, and particularly, for treatments of ocular diseases. Other suitable sEH inhibitors include, for example, trans-4-(4-[3-adamantan-1-yl-ureido]-cyclohexyloxy)-benzoic acid (t-AUCB (5)) and 7-(trifluoromethyl)-N-(4-(trifluoromethyl)phenyl) benzo[d]isoxazol-3-amine (7). Still other suitable inhibitors include, for example, 12-(3-((3s,5s,7s)-adamantan-1-yl)ureido)dodecanoic acid (AUDA); sorafenib; 1-(1-acetyl-piperidin-4-yl)-3-adamantan-1-yl-urea (AR9281); (1R,3S)—N-(4-cyano-2-(trifluoromethyl)benzyl)-3-((4-methyl-6-(methylamino)-1,3,5-triazin-2-yl)amino)cyclohexane-1-carboxamide (GSK2256294); 1-(1-propionylpiperidin-4-yl)-3-(4-(trifluoromethoxy)phenyl)urea (TPPU or UC1770); trans-4-{4-[3-(4-trifluoromethoxy-phenyl)-ureido]-cyclohexyloxy}-benzoic acid (t-TUCB or UC1728); (trifluoromethoxyphenyl-3-(1-propionylpiperidin-4-yl) urea) (TUPS or UC1709); N-[(1S,2R)-2-phenylcyclopropyl]-4-[3-(2-pyridinyl)-1,2,4-oxadiazol-5-yl]-)1-piperidinecarboxamide; and other sEH inhibitors as taught in Shen and Hammock, J Med Chem, 55, 1789-1808 (2012). Other methods of sEH inhibition include, for example, antisense RNA targeting sEH (EPHX2) RNA; shRNA targeting sEH (EPHX2) RNA; siRNA targeting sEH (EPHX2) RNA; RNA silencing targeting sEH (EPHX2) RNA; RNA interference (RNAi) targeting sEH (EPHX2) RNA; CRISPR/Cas9-mediated genetic ablation of sEH (EPHX2) genomic DNA, zinc-finger nuclease-mediated genetic ablation of sEH (EPHX2) genomic DNA, and combinations thereof.

The sEH inhibitors used in the methods of the disclosure can be administered as a pharmaceutical composition comprising the inhibitor of interest in combination with one or more pharmaceutically acceptable carriers. As used herein, the phrase “pharmaceutically acceptable” refers to those ligands, materials, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier”, as used herein, refers to a pharmaceutically acceptable material, formulation or vehicle, such as a liquid or solid filler, diluent, excipient, solvent or encapsulating material, involved in carrying or transporting the active compound from one organ or portion of the body, to another organ or portion of the body. Each carrier must be “acceptable” in the sense of being compatible with the other components of the composition (e.g., synthetic compound) and not injurious to the subject. Lyophilized compositions, which may be reconstituted and administered, are also within the scope of the present disclosure.

Pharmaceutically acceptable carriers may be, for example, excipients, vehicles, diluents, and combinations thereof. For example, where the compositions are to be administered orally, they may be formulated as tablets, capsules, granules, powders, or syrups; or for parenteral administration, they may be formulated as injections (intramuscular, subcutaneous, intramedullary, intrathecal, intraventricular, intravenous, intravitreal), drop infusion preparations, or suppositories. For application by the ophthalmic mucous membrane route, they may be formulated as eye drops or eye ointments. These compositions can be prepared by conventional means, and, if desired, the active compound (i.e., sEH inhibitor) may be mixed with any conventional additive, such as an excipient, a binder, a disintegrating agent, a lubricant, a corrigent, a solubilizing agent, a suspension aid, an emulsifying agent, a coating agent, or combinations thereof.

Suitable dosages of the sEH inhibitors for use in the methods of the present disclosure will depend upon a number of factors including, for example, age and weight of an individual, severity of ocular disease, specific sEH inhibitor to be used, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

In one particularly suitable embodiment, the sEH inhibitor is administered in a dosage ranging from about 0.1 μg to about 300 mg. In one particularly suitable embodiment, the sEH inhibitor is administered oral as a tablet or capsule once a day.

Administration of an effective amount of the sEH inhibitor may be by a single dose, multiple doses, as part of a dosage regimen, and combinations thereof as determined by those skilled in the art for the relevant mechanism or process. The dosage regimen may vary depending on the symptoms, age and body weight of the subject, the nature and severity of the disorder to be treated or prevented, the route of administration and the form of the drug. In one embodiment, the sEH inhibitor is administered via intravitreal injection to the subject, and is administered once a month. In another embodiment, the sEH inhibitor is administered via eye drop or eye ointment to the subject, and is administered once a day. In yet another embodiment, the sEH inhibitor is administered via eye drop or eye ointment to the subject, and is administered twice a day.

It should be understood that the pharmaceutical compositions of the present disclosure can further include additional known therapeutic agents, drugs, modifications of the synthetic compounds into prodrugs, and the like for alleviating, mediating, preventing, and treating the diseases, disorders, and conditions described herein. For example, in one embodiment, the sEH inhibitors can be administered with one or more anti-vascular endothelial growth factor (VEGF) agents, including, but not limited to, pegaptanib, ranibizumab, aflibercept, bevacizumab, brolucizumab (also known as ESBA1008 and RTH258), conbercept (also known as KH-902), Abicipar Pegol, pazopanib, regorafenib, and PAN-90806 and combinations thereof.

The pharmaceutical compositions including the sEH inhibitors and, optionally, additional therapeutic agents and pharmaceutical carriers, used in the methods of the present disclosure can be administered to a subset of subjects in need of treatment for ocular eye disease, including retinopathy of prematurity (ROP), proliferative diabetic retinopathy (PDR), diabetic retinopathy, wet age-related macular degeneration (AMD) pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and the like. Some subjects that are in specific need of treatment for ocular disease may include subjects who are susceptible to, or at elevated risk of, experiencing ocular disease (e.g., retinopathy of prematurity, diabetic retinopathy, “wet” age-related macular degeneration, etc.), and the like. Subjects may be susceptible to, or at elevated risk of, experiencing ocular diseases due to family history, age, environment, and/or lifestyle. Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

Various functions and advantages of these and other embodiments of the present disclosure will be more fully understood from the examples shown below. The examples are intended to illustrate the benefits of the present disclosure, but do not exemplify the full scope of the disclosure.

### Example 1

In this Example, SH-11037 was identified as an inhibitor of soluble epoxide hydrolase (sEH), a key enzyme for the metabolism of ω-3 and ω-6 epoxy fatty acids. Further, sEH levels were analyzed in ocular sections from a choroidal neovascularization (CNV) mouse model and human wet AMD eyes, and sEH was analyzed as a possible target for inhibiting the choroidal neovascularization that underlies wet AMD.

Methods

Preparation of Photoaffinity Reagents

Photoaffinity reagents were synthesized as described in Lee et al., Bioorg. Med. Chem. Lett. 26, 4277-4281, with purity confirmed as >95% by HPLC. For pulldowns, Neutravidin agarose beads (1 mL of 50% slurry) were washed three times in buffer A (25 mM Tris-HCl pH 7.4, 150 mM NaCl, 2.5 mM sodium pyrophosphate, 1 mM phenylmethylsulfonyl fluoride (PMSF), 0.1 mM sodium orthovanadate, 10 μg/mL aprotinin and 10 μg/mL leupeptin). The beads were then incubated with 75 μM affinity reagents 2, 3, or 4 dissolved in DMSO (final DMSO concentration: <0.1% v/v) and diluted in this buffer, overnight at 4° C. with rotation. The beads were blocked using 1 mM biotin solution prepared in buffer A for 1 hour followed by incubation with 1 mg/mL cytochrome c solution for 1 hour at 4° C. The beads were then washed three times with buffer A and resuspended in 1 mL.

Photoaffinity Pulldown Experiments

Flash-frozen porcine brain (20 g) obtained from the Purdue-Indiana University School of Medicine Comparative Medicine Program was homogenized in 50 mL buffer A using a tissue homogenizer. The homogenate was centrifuged and sonicated. The lysate was then centrifuged at 11,000×g for 30 minutes. The resulting supernatant (S2) was collected. The pellet (P1) was resuspended in buffer A and centrifuged at 11,000×g for 30 minutes; supernatant (S3) was collected. Both S2 and S3 supernatants were divided equally and each fraction was incubated with 500 μL photoaffinity or control reagent conjugated to Neutravidin beads for 75 minutes at 4° C. with shaking. The beads were collected by centrifugation then resuspended in buffer A+1% (v/v) Triton X-100 and irradiated with 365 nm UV light (Mercury bulb H44GS100 from Sylvania in a Blak-Ray 100A long-wave UV lamp with output of 25 mW/cm² at sample distance) for 30 minutes at 4° C. The beads were then washed in high-salt buffer containing 25 mM Tris-HCl pH 7.4, 350 mM NaCl, 1% (v/v) Triton X-100 and 1 mM PMSF. The beads were washed again in salt-free buffer containing 25 mM Tris-HCl, 1% (v/v) Triton X-100 and 1 mM PMSF. After 5 minutes of incubation, the beads were collected, then boiled in SDS-PAGE gel loading dye containing 2-mercaptoethanol for 10 minutes at 70° C. to release the bound proteins. After boiling, the contents were allowed to cool and after a quick spin the eluate was collected using a Hamilton syringe. The eluates were then analyzed in 4-20% (w/v) gradient SDS-PAGE and the protein bands were visualized using silver staining. The protein bands pulled down specifically by photoaffinity reagent were excised from the silver stained SDS-PAGE gel and analyzed by mass spectrometry (IUSM Proteomics Core). Using SEQUEST™ algorithms and the swine database (UniProt), the identities of the pulled down proteins were confirmed.

Immunoblot Assay

Cell lysates were prepared by homogenizing retina and choroid in NP-40 lysis buffer (25 mM HEPES pH 7.6, 150 mM NaCl, 1% (v/v) NP-40, 10% (v/v) glycerol, 1 mM sodium orthovanadate, 10 mM NaF, 1 mM PMSF, 10 μg/mL aprotinin, 1 μM pepstatin, 1 μM leupeptin) and then centrifuged at 12,000×g for 15 minutes at 4° C. Supernatant was collected and protein concentration was determined using a Bradford assay. Equal amounts of total protein (40 μg) from each sample were resolved by 10% (w/v) SDS-PAGE and then transferred onto PVDF membranes. Proteins were immunoblotted with antibodies against sEH (H215) (Santa Cruz) at 1:1000 dilution, and (3-actin (AC40) (Sigma-Aldrich) at 1:5000. Secondary antibodies (Thermo Fisher Scientific) were used at 1:10,000 dilutions. All of the dilutions were made in Tris Buffered Saline-0.05% (v/v) Tween-20 buffer containing 2% (w/v) bovine serum albumin (BSA). Signals were detected using Amersham ECL immunoblotting detection reagents on a Typhoon molecular imager (GE Healthcare).

Recombinant Soluble Epoxide Hydrolase Activity Assay

Small molecule inhibition of soluble epoxide hydrolase (sEH) enzymatic activity was evaluated using a fluorimetric sEH inhibitor screening assay kit based on the synthetic, fluorogenic substrate PHOME (3-phenyl-cyano(6-methoxy-2-naphthalenyl)methyl ester-2-oxiraneacetic acid) (Cayman Chemical, Ann Arbor, Mich.) following manufacturer's instructions, using varying concentrations of SH-11037 (1), t-AUCB (5) (Cayman Chemical, Ann Arbor, Mich.), and 6 (synthesized as described in (Basavarajappa, et al., (2015) J. Med. Chem. 58, 5015-5027)). Benzisoxazole sEH inhibitor 7 was synthesized according to a published method, and characterization matched published parameters. (Shen, et al., (2009) Bioorg. Med. Chem. Lett. 19, 5716-5721) Purity of all synthesized compounds was >95% by HPLC. Compounds were dissolved in DMSO (final DMSO concentration=5% (v/v)). Activity was calculated according to:

% Initial Activity=[(F_{I15}−F_{I0})/(F_{T15}−F_{T0})]×100%

where F_{I} is the background corrected fluorescence signal obtained in the presence of an inhibitor and F_{T} is the background corrected fluorescence signal obtained for the total activity at times 0 and 15 minutes. IC₅₀ values were calculated using GraphPad Prism v. 7.0.

sEH Enzyme Kinetics

Various concentrations of compound dissolved in DMSO (final DMSO concentration=5% (v/v)) and human sEH (60 ng/mL final concentration; Cayman Chemical) in 25 mM bis-Tris-HCl buffer containing 0.1% (w/v) BSA were mixed in a 96 well plate. PHOME at indicated concentrations was added to the wells to initiate the reaction and fluorescence was read with an excitation wavelength of 330 nm and emission wavelength of 465 nm. The standard curve plotted from dilutions of the product, 6-methoxy-2-naphthaldehyde, was used to convert fluorescence reading (RFU/min) to sEH activity (nmol product formed/min). The reaction rate was obtained from the slope of the line from time=5 to 15 minutes for each substrate concentration, and analyzed using GraphPad Prism 7 and SigmaPlot 13.0 to determine Michaelis-Menten kinetic parameters.

Mice

All mouse experiments followed the guidelines of the Association for Research in Vision and Ophthalmology Statement for the Use of Animals in Ophthalmic and Visual Research and were approved by the Indiana University School of Medicine Institutional Animal Care and Use Committee. Wild-type female C57BL/6J mice, 6-8 weeks of age, were purchased from the Jackson Laboratory (Bar Harbor, Me.). Intraperitoneal injections of 17.5 mg/kg ketamine hydrochloride and 2.5 mg/kg xylazine mixture were used for anesthesia. At the end of the experiments, mice were euthanized by carbon dioxide asphyxiation followed by cervical dislocation.

Laser-Induced Choroidal Neovascularization

The L-CNV mouse model and injections were performed as previously described. (Sulaiman, et al., (2015), J. Ocul. Pharmacol. Ther. 31, 447-454) Briefly, eyes were dilated using 1% (w/v) tropicamide, then underwent laser treatment using 50 μm spot size, 50 ms duration, and 250 mW pulses of an ophthalmic argon green laser, wavelength 532 nm, coupled to a slit lamp. Compounds where indicated were injected a single time immediately post-laser treatment, delivered intravitreally using a 33-gauge needle, in a 0.5 μL volume. SH-11037, t-AUCB, or 7 were dissolved in DMSO then diluted in PBS to a final concentration of 0.5% (v/v) DMSO. Vehicle alone (PBS+0.5% (v/v) DMSO) was used as negative control. Mouse anti-VEGF₁₆₄ antibody (AF-493-NA, R&D Systems) at a 5 ng dose was used as a positive control for inhibition of neovascularization. Eyes were numbed with tetracaine solution before the injection, and triple antibiotic ointment was used immediately after the injection to prevent infection.

Lipid Profiling

C57BL/6J mice underwent laser treatment followed by intravitreal injections of 10 μM SH-11037, 10 μM t-AUCB or vehicle control as described above. Mice were sacrificed 3 days post-laser-treatment, eyes were enucleated and retina/choroid layers were immediately separated and stored at −80° C. Lipid profile analysis was performed by the Lipidomics Core Facility at Wayne State University using standard operating procedures developed by the core as previously described. (Maddipati, et al. (2011), Prostaglandins Other Lipid Mediators 94, 59-72)

Immunohistochemistry

Eyes from L-CNV and control mice fixed in 4% (w/v) PFA overnight were paraffin embedded and sectioned. Human donor eyes from wet AMD patients or age-matched controls with no documented ocular pathology were obtained from the National Disease Research Interchange (NDRI; Philadelphia, Pa.) with full ethical approval for use in research. All sample tissues were anonymized prior to receipt in the laboratory. Eye sections were deparaffinized, rehydrated and underwent heat induced antigen retrieval. Sections were washed in TBS and blocked in 10% (v/v) DAKO diluent in TBST/1% (w/v) BSA for an hour at room temperature, then incubated with primary antibodies overnight at 4° C. Primary antibodies and dilutions used were rabbit anti-sEH, H215 (1:250 for mouse, 1:90 for human; Santa Cruz); mouse anti-sEH, A5 (1:250; Santa Cruz); Ricinus communis agglutinin I (rhodamine labeled, 1:250 for mouse; FITC-labeled, 1:400 for human; Vector Labs); mouse anti-rhodopsin, ab3424 (1:300; Abcam); rabbit anti-cone arrestin, AB15282 (1:500; Millipore); mouse-anti-calbindin, ab11426 (1:300, Abcam); rabbit anti-Brn3a, AB5945 (1:400; Millipore); and rabbit anti-vimentin PLA0199 (1:300; Sigma) diluted in 10% (v/v) DAKO diluent in TBST/1% (w/v) BSA (only PBS/1% (w/v) BSA for human). Sections were then incubated with secondary antibodies, Alexa Fluor 488 (647 for human) goat anti-rabbit and Alexa Fluor 555 goat anti-mouse (Abcam) diluted 1:400 (1:500 for human) in 10% (v/v) DAKO diluent in TBST/1% (w/v) BSA (only PBS/1% (w/v) BSA for human) for 45-60 minutes at room temperature, followed by a brief wash in TBS, dehydration through an ethanol series and mounting with Vectashield mounting medium with DAPI (Vector Labs). Images were acquired with an LSM700 confocal microscope (Zeiss) with a 20× objective or, for human sections, an Axiolmager D2 (Zeiss).

Tissue-Based sEH Activity Assay Using Trans-Stilbene Oxide

The sEH activity in tissue homogenates was assayed using trans-stilbene oxide (t-SO) as substrate. The assay is based on the hydrolysis of t-SO that is tracked as a decrease in the absorbance at 230 nm. L-CNV was induced as described above. After 3 days, enucleated eyes from both untreated and L-CNV mice were homogenized in 0.2 M sodium phosphate buffer, pH 7.4. In order to remove microsomal epoxide hydrolase and lenses, tissue extracts were centrifuged at 100,000×g for 30 minutes at 4° C. After protein estimation, 100 μL of tissue homogenates (100 μg/mL) and 98 μL of SH-11037 or 7 dissolved in DMSO/buffer (final 1% (v/v) DMSO) were added to a UV-transparent 96-well plate. After 5 minutes' incubation at room temperature, 2 μL of t-SO in ethanol (100 μM final concentration) was added to assay wells to initiate the reaction. The absorbance was read at 230 nm for 20 minutes. sEH activity was determined as follows:

sEH Activity=[(A₀−A₂₀)−(B₀−B₂₀)]/mg of protein in a reaction

Where A₀ and A₂₀ are absorbance of test wells read at 230 nm at time 0 and 20 minutes respectively, and B₀ and B₂₀ are absorbance of background wells read at 230 nm at time 0 and 20 minutes.

Choroidal Flatmount and Analysis.

To assess neovascularization in response to treatments, 14 days post-L-CNV induction, eyes were enucleated, fixed and stained as described. (Sulaiman, et al., (2016), Sci. Rep. 6, 25509) Choroid/sclera layers were incubated with rhodamine labeled Ricinus communis agglutinin I (Vector Labs), in the dark for 45 minutes, to stain blood vessels. Flatmounts of the choroid were mounted with Vectashield mounting medium (Vector Labs) and Z-stack images were taken on an LSM700 confocal microscope (Zeiss). ImageJ software was used to analyze Z-stack images. This experiment was performed by a masked investigator and followed the guidelines and exclusion criteria described previously, (Poor, et al., (2014), Invest. Ophthalmol. Visual Sci. 55, 6525-6534) to ensure reproducibility and eliminate investigator bias.

Choroidal Sprouting Assay

Benzisoxazole sEH inhibitor 7 was synthesized according to the method as described in Shen et al., Bioorg. Med. Chem. Lett. 119, 5716-5721. Sprouting of endothelial cells from choroidal layers was tested as described in Sulaiman et al., Sci. Rep. 6, 25509. t-AUCB or 7, dissolved in DMSO, were tested at 0.1, 1, and 10 μM concentrations for 48 hours. The final concentration of DMSO in each well was 0.2% (v/v). Images were taken using an EVOS-fl digital microscope (AMG, Mill Creek, Wash., USA) and data were analyzed as the sprouting distance in four different directions using ImageJ software v.1.48v (http://imagej.nih.gov/ij/).

Statistical Analyses

Statistical analyses were performed with GraphPad Prism 7 software. One-way ANOVA was used with Tukey's post hoc test for lipid profiling, and Dunnett's post hoc test for L-CNV confocal analysis and choroidal sprouting experiments. Two-sided P values <0.05 were considered statistically significant.

Results

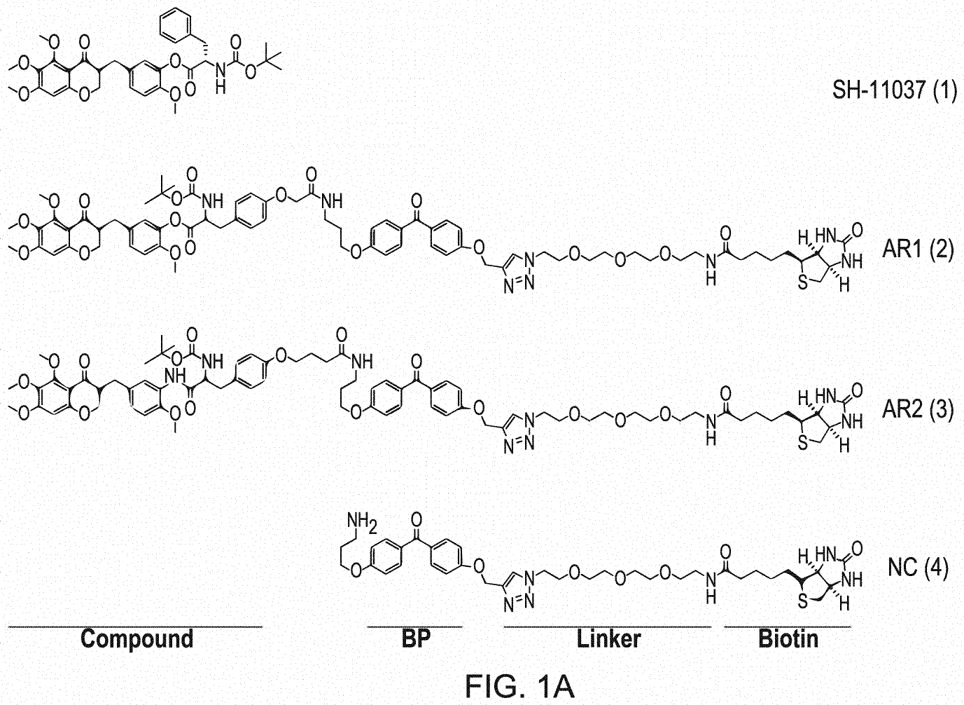

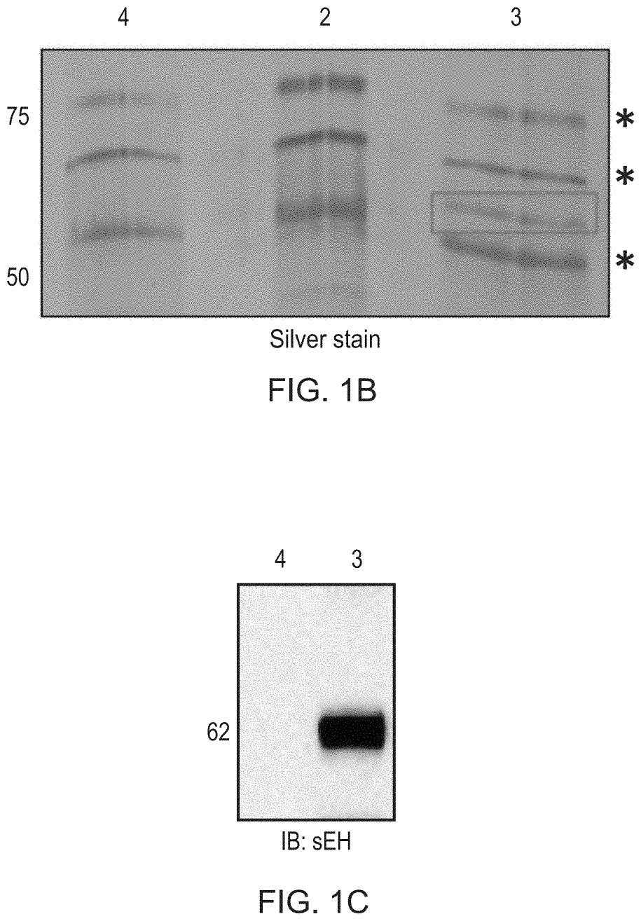

SH-11037 protein targets were identified using an unbiased forward chemical genetics approach. First, two photoaffinity reagents 2 and 3 were synthesized that retained antiangiogenic activity, and a control compound 4 was also synthesized (FIG. 1A). The ester group in 2 (shared with SH-11037) was replaced by an amide in 3 for increased stability. The SH-11037-based affinity reagents 2 and 3 were immobilized used to pull down protein binding partners from a porcine brain lysate. Affinity reagent 3, but not the negative control reagent 4, pulled down a specific protein target, which was identified using mass spectrometry to be soluble epoxide hydrolase (FIG. 1B). Immunoblot confirmed the identity of the pulled down protein using an sEH specific antibody (FIG. 1C).

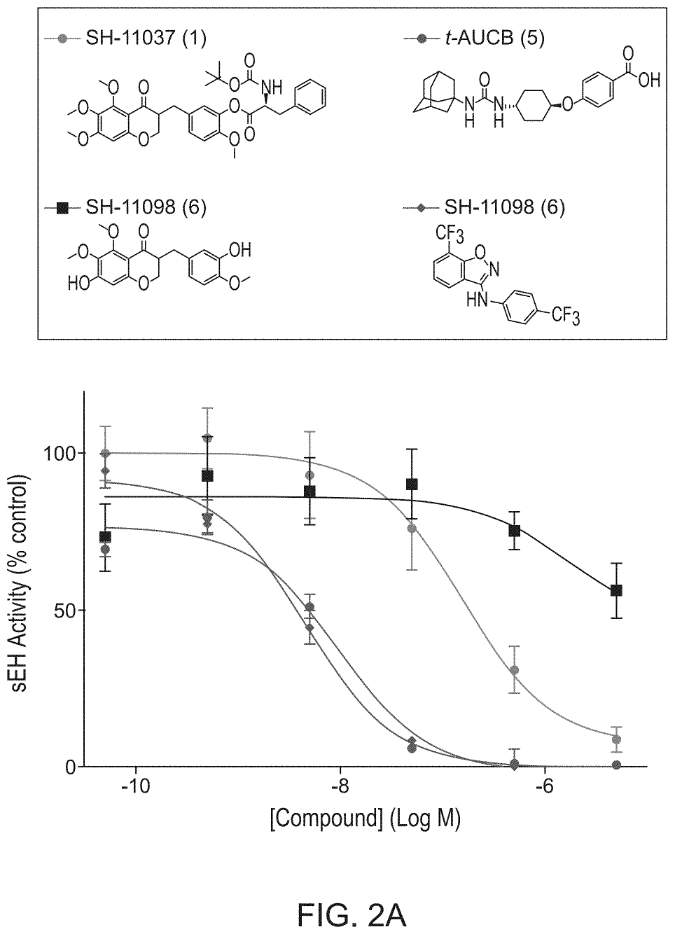

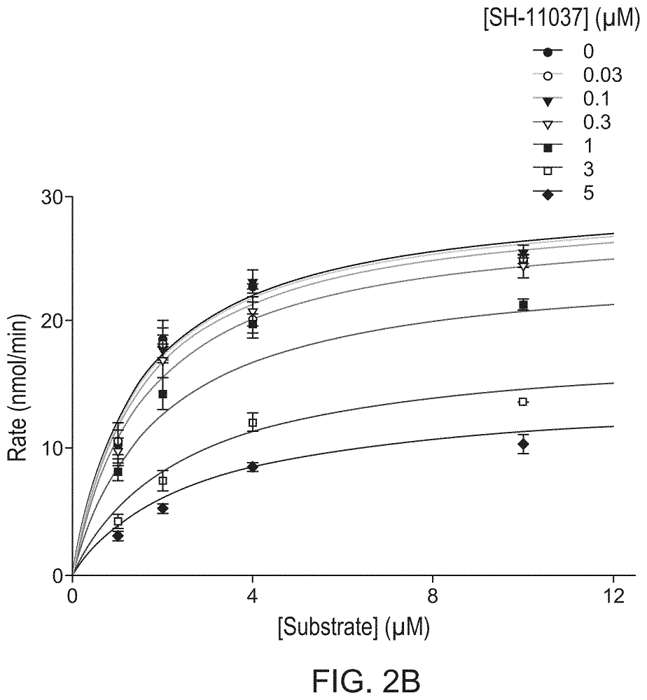

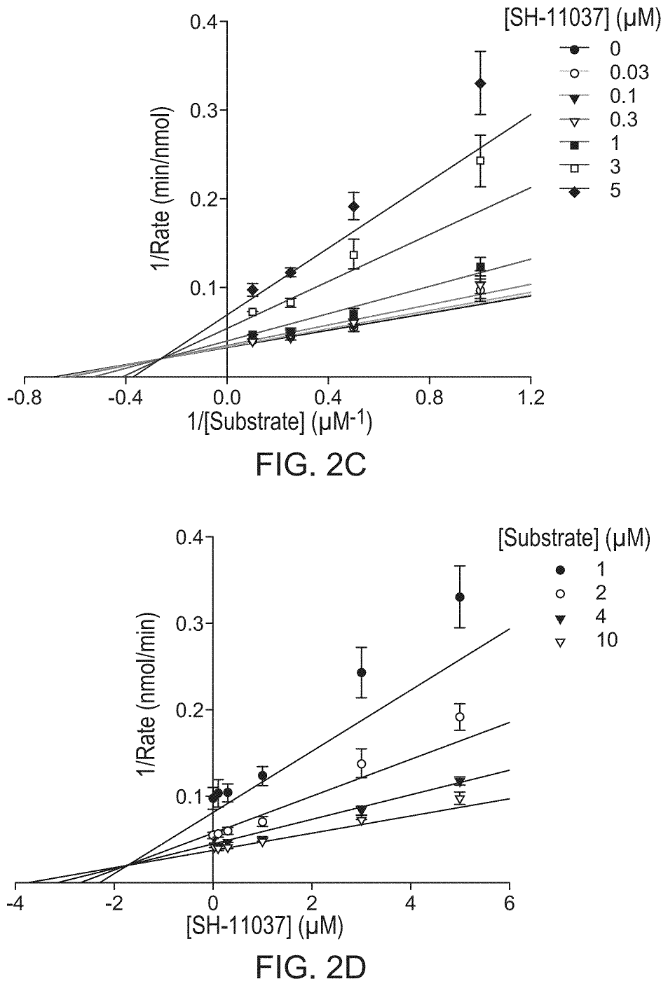

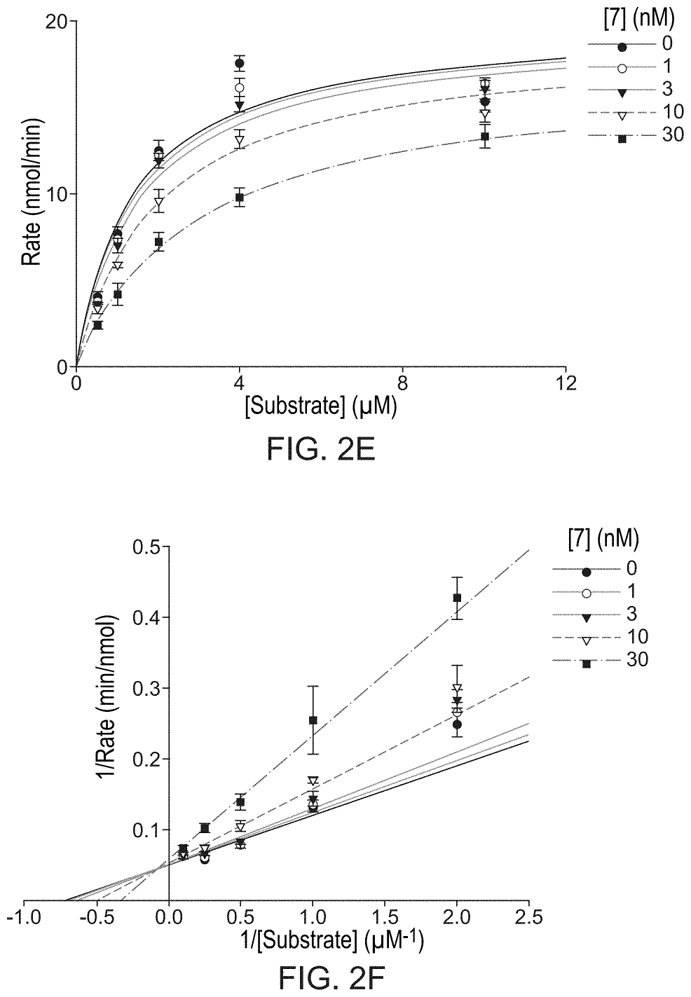

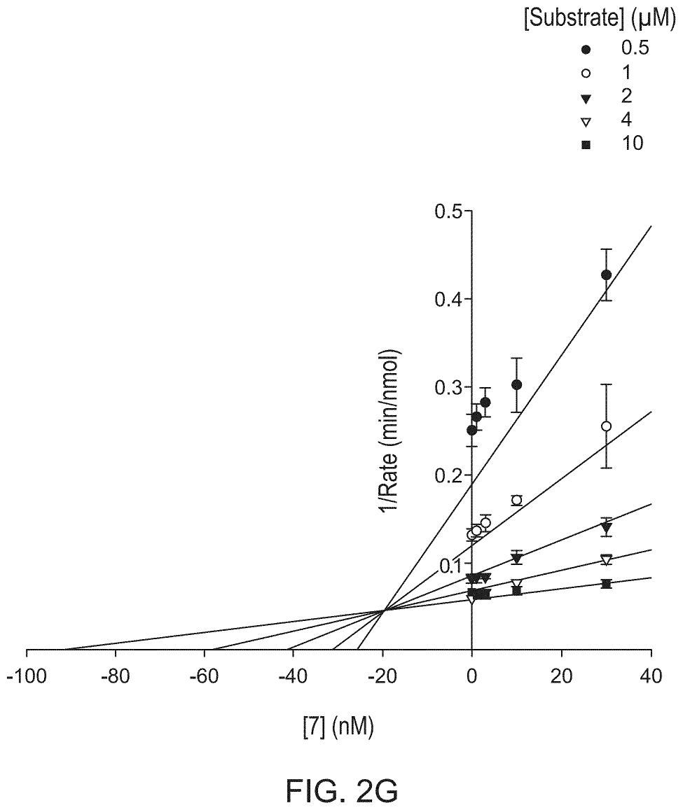

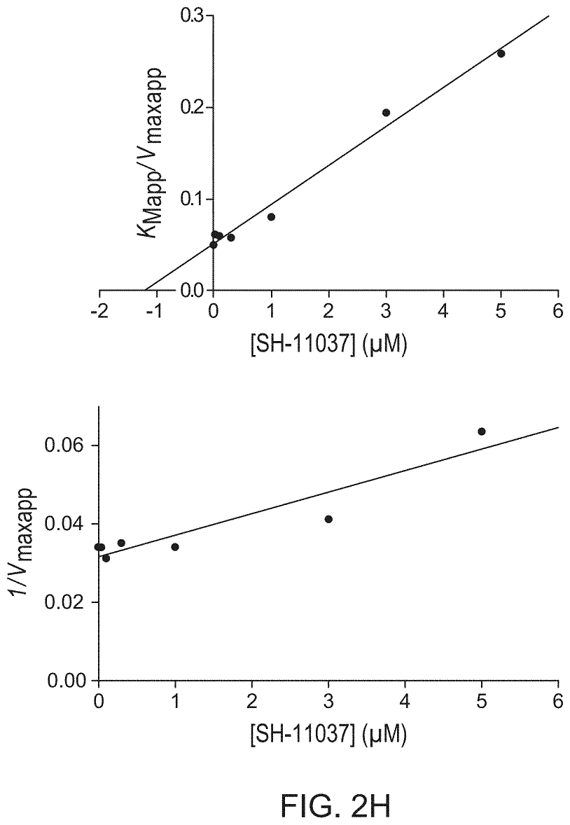

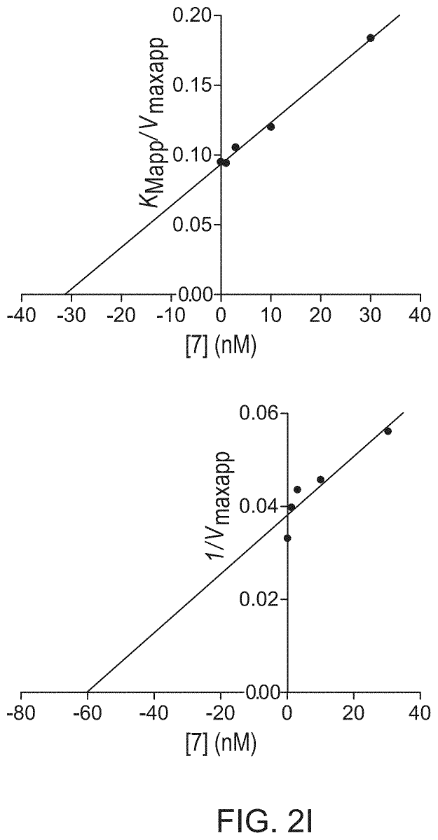

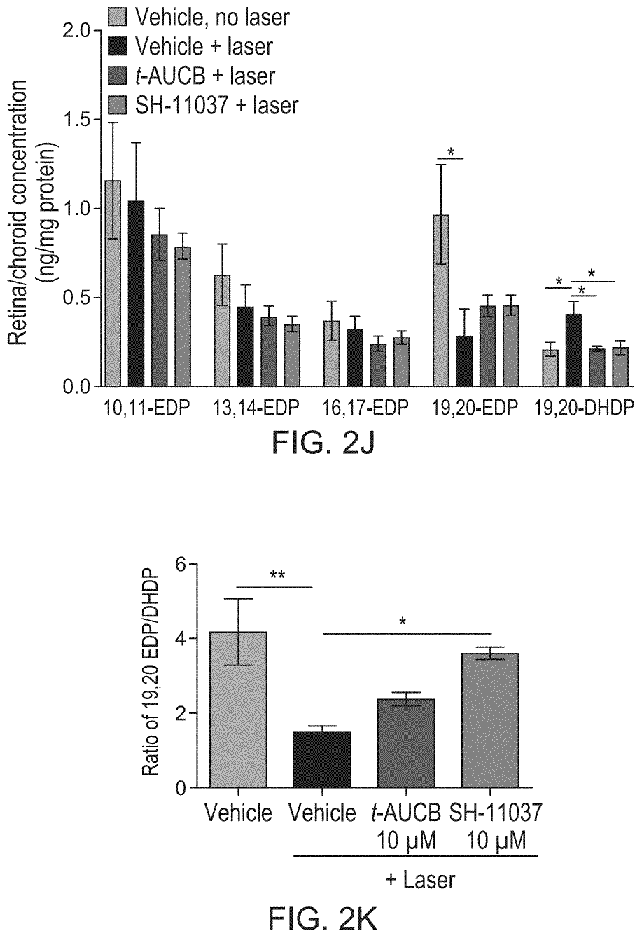

Following the identification of sEH as a binding target of SH-11037, it was analyzed whether SH-11037 would interfere with the epoxide hydrolase activity of sEH in vitro, compared to a positive control, a known sEH inhibitor. t-AUCB (5) (FIG. 2A) is a specific inhibitor of the epoxide hydrolase activity of sEH. It has been the most commonly used sEH inhibitor in preclinical studies due to its high solubility and potency with limited toxicity. Meanwhile, 7-(trifluoromethyl)-N-(4-(trifluoromethyl)phenyl) benzo[d]isoxazol-3-amine (7) is a structurally distinct benzisoxazole inhibitor with excellent potency and pharmacokinetic properties. Interestingly, SH-11037 inhibited sEH enzymatic activity in vitro in a concentration-dependent manner (FIG. 2A), although not as potently as t-AUCB or (7).

To test whether these effects are specific to SH-11037, a negative control compound, SH-11098 (6), which is a homoisoflavonoid that was found to be inactive in angiogenesis assays in vitro was also tested. This related compound had minimal effect on sEH activity, suggesting that structural features of SH-11037 specifically interact with this enzyme.

Enzyme kinetics analysis showed that increasing concentrations of SH-11037 decreased Vₘₐₓ and increased K_{M}, revealing that SH-11037 is a mixed-type inhibitor of sEH (FIGS. 2B-2D), with Kᵢ=1.73±0.45 μM. Compound (7) is also a mixed-type inhibitor (FIGS. 2E-2G). Furthermore, secondary plots of K_{Mapp}/Vₘₐₓₐₚₚ and 1/Vₘₐₓₐₚₚ vs [SH-11037] and (7) fit the curves expected for mixed-type inhibition (FIGS. 2H & 2I). The catalytic mechanism of sEH proceeds as a nucleophilic attack onto the epoxide substrate by an Asp residue, which results in a tetrahedral intermediate, requiring activated water to release the diol and regenerate free enzyme. Given that sEH has two substrates (e.g., fluorogenic substrate 3-phenyl-cyano(6-methoxy-2-naphthalenyl)methyl ester-2-oxiraneacetic acid [PHOME] and water) and involves a covalent intermediate, it is possible that SH-11037 may bind and stabilize an enzyme species late in the catalytic cycle that is still in conformation equilibrium with the free enzyme. SH-11037 binding in the active site of such an enzyme species may not compete with the substrates. Taken together, these findings indicate that SH-11037 represents a novel, distinct chemotype from known sEH inhibitors. In particular, it lacks the urea moiety seen in a majority of active structures.

After establishing SH-11037's binding and in vitro inhibition of sEH activity, it was crucial to assess whether the previously-documented antiangiogenic effects of sEH were mediated through the inhibition of sEH in the L-CNV model. Therefore, the lipid profiles of the retina/choroid layers from mice were analyzed three days after CNV induction and intravitreal injections of 10 μM SH-11037 or t-AUCB. Since DHA is the most abundant bioactive lipid in the eye, DHA epoxy and dihydroxy metabolite levels were evaluated to investigate sEH activity in vivo. Interestingly, 19,20-epoxydocosapentaenoic acid (EDP) and its dihydroxy metabolite, 19,20-dihydroxydocosapentaenoic acid (DHDP) appeared to be the assessed DHA metabolites most affected by sEH inhibition (FIG. 2J). Moreover, the ratio of 19,20-EDP to 19,20-DHDP, which decreased after induction of neovasularization, indicative of enhanced sEH activity under these conditions. However, this ratio was partially normalized after SH-11037 or t-AUCB treatment compared to the vehicle treated controls, indicating sEH inhibition in vivo (FIG. 2K). Despite being less potent than t-AUCB in vitro (FIG. 2A), SH-11037 performed comparably in vivo (FIG. 2K), perhaps indicative of better ocular bioavailability than the existing inhibitor.

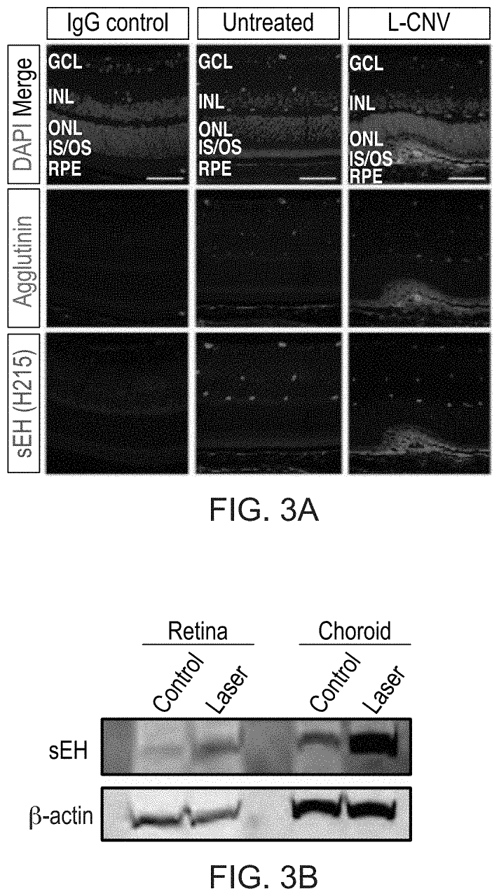

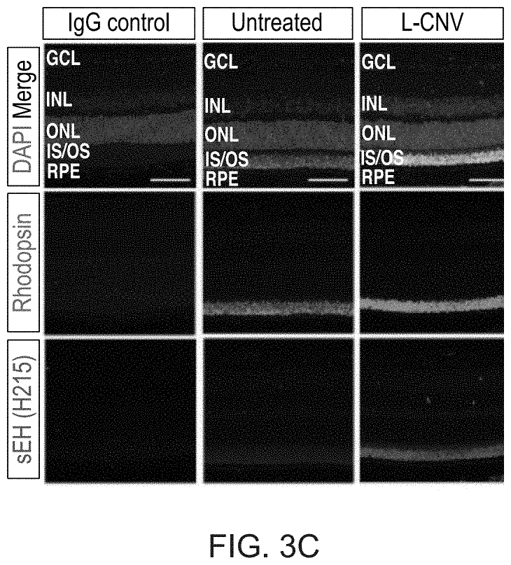

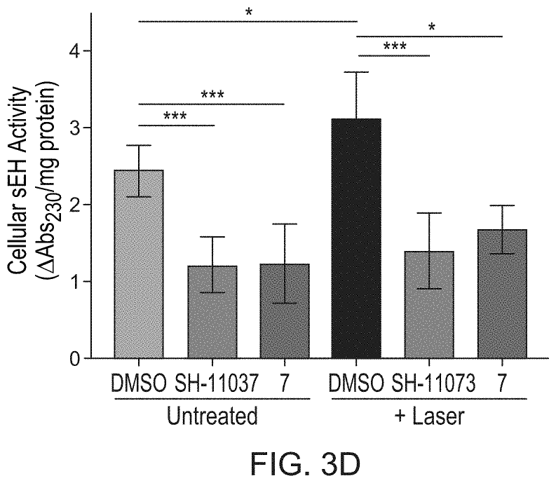

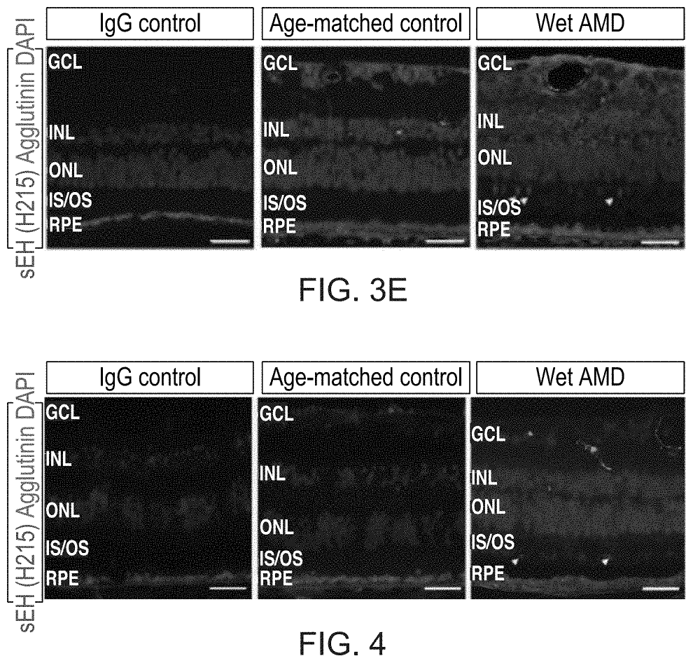

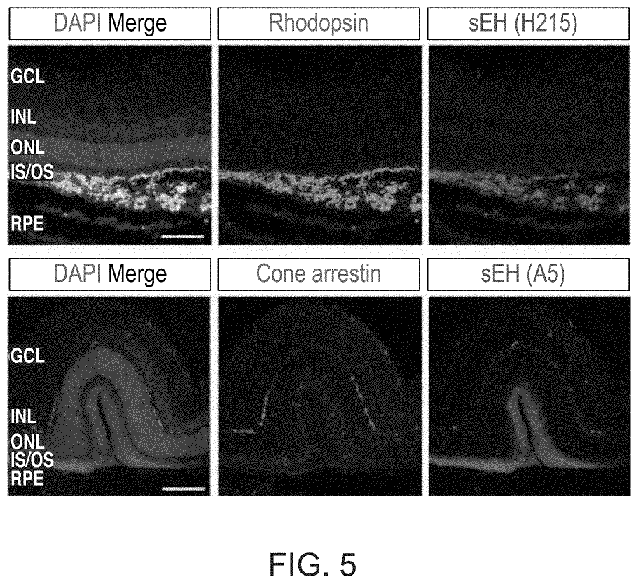

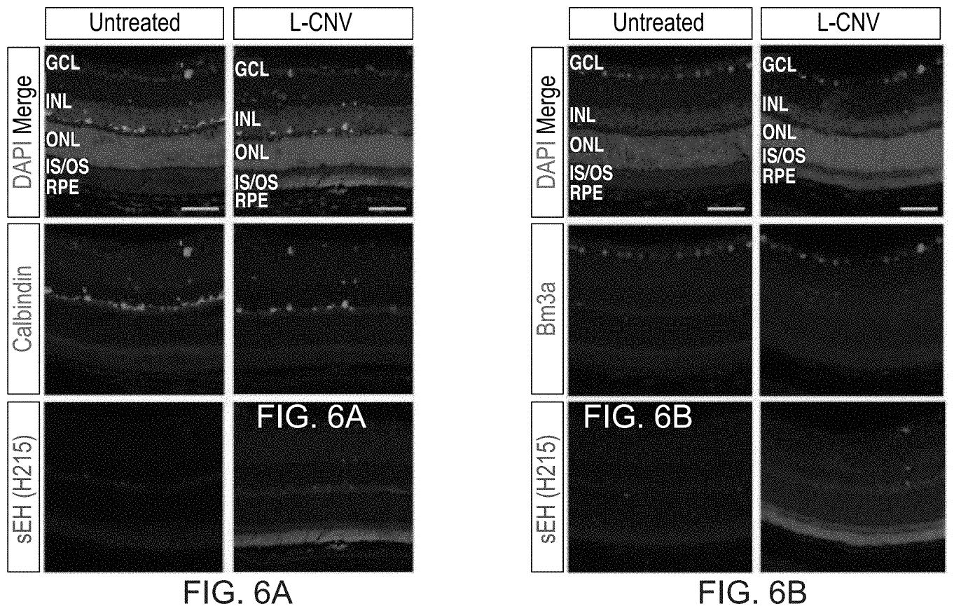

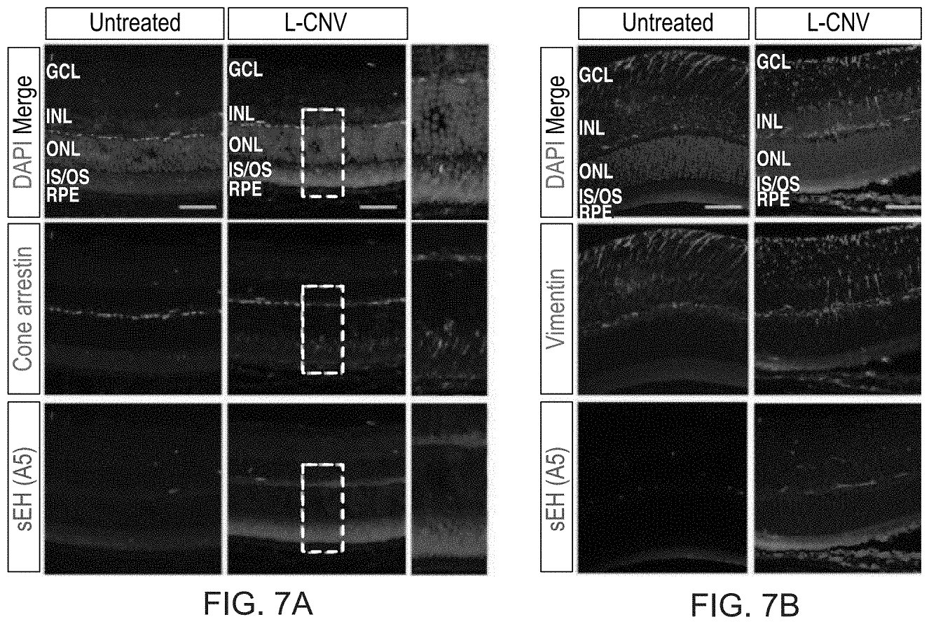

Given the significantly suppressed ratio of 19,20 EDP/DHDP after laser induction of CNV compared to the untreated control, suggestive of increased sEH activity, it was tested whether there were differences in sEH expression during neovascularization in the L-CNV model. Intriguingly, L-CNV treated mice demonstrated a substantial upregulation of sEH levels in photoreceptor layers, both within and surrounding the neovasular lesion, compared to untreated eyes (FIG. 3A). This upregulation of sEH in L-CNV was further confirmed in immunoblots of retina and choroid layers of laser treated mouse eyes relative to untreated controls, suggesting a role for sEH in the L-CNV model (FIG. 3B). Co-immunostaining revealed co-localization of upregulated sEH levels with rod photoreceptors in the eyes of L-CNV mice compared to controls (FIG. 3C, FIG. 5), but no overlap with markers of other retinal cell types, including retinal ganglion cells, horizontal cells, Müller glia, and cone photoreceptors (FIGS. 5-7). This increase in immunostaining corresponded to an increase in sEH activity in L-CNV eye lysates, which could be normalized by SH-11037 or compound 7 treatment (FIG. 3D). Surprisingly, postmortem human wet AMD patients' eyes also revealed changed sEH expression in the central retina compared to age-matched controls: an increase in the staining pattern of sEH in the inner retina seen in age-matched control retina, and aberrant expression in some photoreceptors (FIG. 3E). Together, these data strongly suggest a role for sEH in the CNV process both in mice and humans.

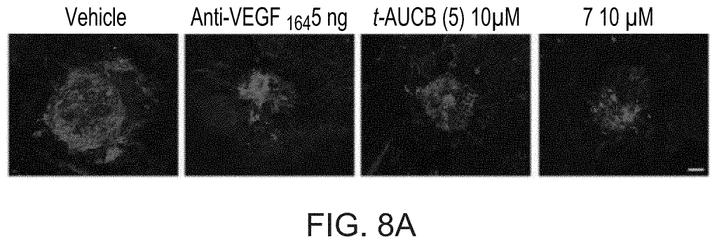

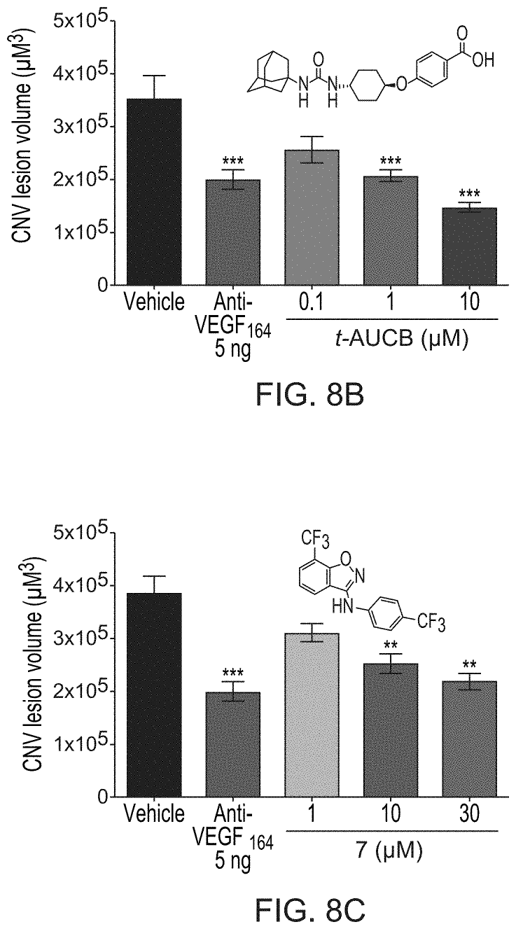

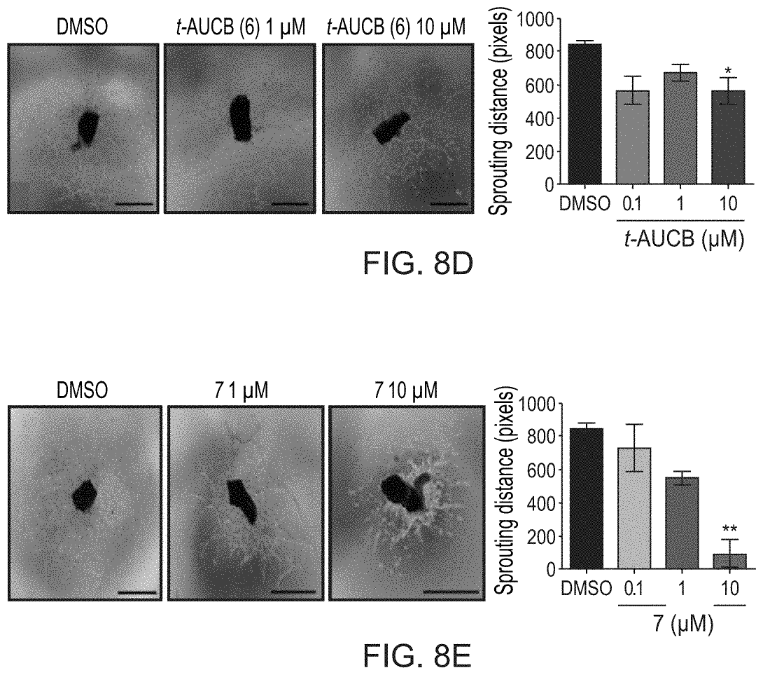

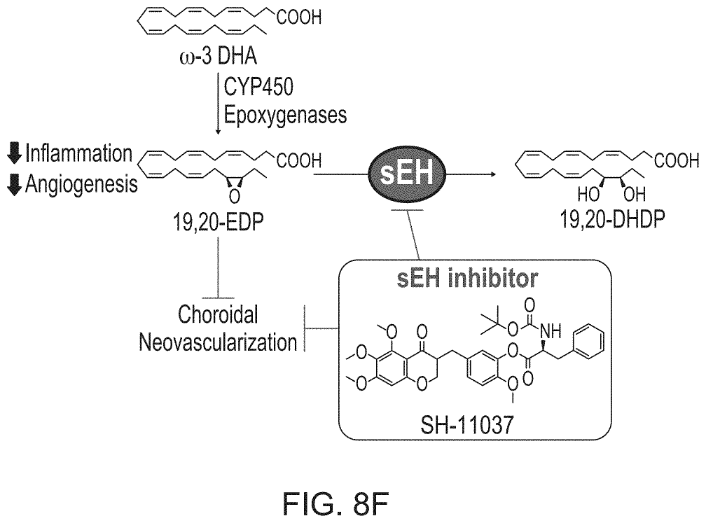

sEH inhibitors were tested locally, using intravitreal injections, to minimize any systemic side effects and focus on understanding the effects of sEH in the eye specifically. It was previously shown that SH-11037 was effective at doses ≥1 μM in this context (Sulaiman et al., (2016), Sci. Rep. 6, 25509; WO 2017/091473 assigned to Indian University Research & Technology Corporation, Jun. 1, 2017). Here, to validate sEH as a key target, the antiangiogenic effect of two chemically distinct small molecule inhibitors of sEH, t-AUCB (5) and compound 7 were assessed in L-CNV (FIGS. 8A-8E). A single injection of either t-AUCB or compound 7 dose-dependently suppressed CNV lesion vascular volume compared to vehicle (FIGS. 8A-8C) and comparable to the standard of care-equivalent anti-VEGF₁₆₄ antibody, suggesting indeed that sEH inhibition directly in the eye does not require ω-3 supplementation for antiangiogenic efficacy.

Additionally, in order to confirm the observations in a different model system, t-AUCB and compound 7 were tested in the choroidal sprouting assay, as an ex vivo model of CNV. Interestingly, both t-AUCB and compound 7 suppressed the ability of choroidal tissues to form sprouts (FIGS. 8D & 8E). Local small molecule inhibition of sEH is an appealing therapeutic approach of significant interest for wet AMD patients to augment DHA epoxy metabolite levels with or without dietary supplementation of ω-3 PUFA (FIG. 8F).

In conclusion, the above findings reveal not only the target of an antiangiogenic molecule and a novel chemotype for sEH inhibition but also a central role for local sEH in ocular diseases. sEH-targeted therapy is a possible approach to complement or combine with the existing anti-VEGF medications to overcome their limitations and tackle multiple angiogenesis signaling pathways for improved treatment of wet AMD. It has already been shown that SH-11037 can synergize with an anti-VEGF antibody in L-CNV (WO 2017/091473 assigned to Indiana University Research & Technology Corporation, Jun. 1, 2017).

What is claimed is:

1. A method of inhibiting ocular disease in a subject in need thereof, the method comprising administering to the subject a soluble epoxide hydrolase (sEH) inhibitor selected from the group consisting of 7-(trifluoromethyl)-N-(4-(trifluoromethyl)phenyl) benzo[d]isoxazol-3-amine (7); 12-(3-((3s,5s,7s)-adamantan-1-yl)ureido)dodecanoic acid (AUDA); sorafenib; 1-(1-acetyl-piperidin-4-yl)-3-adamantan-1-yl-urea (AR9281); (1R,3S)—N-(4-cyano-2-(trifluoromethyl)benzyl)-3-((4-methyl-6-(methylamino)-1,3,5-triazin-2-yl)amino)cyclohexane-1-carboxamide (GSK2256294); trans-4-{4-[3-(4-trifluoromethoxy-phenyl)-ureido]-cyclohexyloxy}-benzoic acid (t-TUCB or UC1728); N-[(1S,2R)-2-phenylcyclopropyl]-4-[3-(2-pyridinyl)-1,2,4-oxadiazol-5-yl]-)1-piperidinecarboxamide; antisense RNA targeting sEH (EPHX2) RNA; shRNA targeting sEH (EPHX2) RNA; siRNA targeting sEH (EPHX2) RNA; RNA silencing targeting sEH (EPHX2) RNA; RNA interference (RNAi) targeting sEH (EPHX2) RNA; CRISPR/Cas9-mediated genetic ablation of sEH (EPHX2) genomic DNA; zinc-finger nuclease-mediated genetic ablation of sEH (EPHX2) genomic DNA; and combinations thereof.

2. The method as set forth in claim 1 comprising administering from about 0.1 μg to about 300 mg sEH inhibitor to the subject.

3. The method as set forth in claim 1 comprising orally administering the sEH inhibitor to the subject.

4. The method as set forth in claim 1 comprising administering the sEH inhibitor via intravitreal injection once a month to the subject.

5. The method as set forth in claim 1 comprising administering the sEH inhibitor via eye drops or eye ointment at a dosing regimen selected from the group consisting of once a day and twice a day to the subject.

6. The method as set forth in claim 1 further comprising administering an anti-vascular endothelial growth factor (anti-VEGF) agent in combination with the sEH inhibitor.

7. The method as set forth in claim 1, wherein the subject has a disease selected from the group consisting of retinopathy of prematurity (ROP), proliferative diabetic retinopathy (PDR), diabetic retinopathy, wet age-related macular degeneration (AMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and combinations thereof.

8. A method of treating wet age-related macular degeneration (AMD) in a subject, the method comprising administering to the subject a soluble epoxide hydrolase (sEH) inhibitor selected from the group consisting of 7-(trifluoromethyl)-N-(4-(trifluoromethyl)phenyl) benzo 1M isoxazol-3-amine (7); 12-(3-((3s,5s,7s)-adamantan-1-yl)ureido)dodecanoic acid (AUDA); sorafenib; 1-(1-acetyl-piperidin-4-yl)-3-adamantan-1-yl-urea (AR9281); (1R,3S)—N-(4-cyano-2-(trifluoromethyl)benzyl)-3-((4-methyl-6-(methylamino)-1,3,5-triazin-2-yl)amino)cyclohexane- 1-carboxamide (GS K2256294); trans-4-{4-[3-(4-trifluoromethoxy-phenyl)-ureido]-cyclohexyloxy}-benzoic acid (t-TUCB or UC1728); N-[(1S,2R)-2-phenylcyclopropyl]-4-[3-(2-pyridinyl)-1,2,4-oxadiazo 1-5-yl]-)1-piperidinecarboxamide; antisense RNA targeting sEH (EPHX2) RNA; shRNA targeting sEH (EPHX2) RNA; siRNA targeting sEH (EPHX2) RNA; RNA silencing targeting sEH (EPHX2) RNA; RNA interference (RNAi) targeting sEH (EPHX2) RNA; CRISPR/Cas9-mediated genetic ablation of sEH (EPHX2) genomic DNA; zinc-finger nuclease-mediated genetic ablation of sEH (EPHX2) genomic DNA; and combinations thereof.

9. The method as set forth in claim 8 comprising administering from about 0.1 μg to about 300 mg sEH inhibitor to the subject.

10. The method as set forth in claim 8 comprising orally administering the sEH inhibitor to the subject.

11. The method as set forth in claim 8 comprising administering the sEH inhibitor via intravitreal injection once a month to the subject.

12. The method as set forth in claim 8 comprising administering the sEH inhibitor via eye drops or eye ointment at a dosing regimen selected from the group consisting of once a day and twice a day to the subject.

13. The method as set forth in claim 8 further comprising administering an anti-vascular endothelial growth factor (anti-VEGF) agent in combination with the sEH inhibitor.
